(19) United States
(12) Patent Application Publication
(10) Pub. No.: US 2005/0004660 A1
(43) Pub. Date: Jan. 6, 2005

(54) Methods, materials and apparatus for deterring or preventing endoleaks following endovascular graft implantation

(75) Inventors: Robert F. Rosenbluth, Laguna Niguel, CA (US); Brian J. Cox, Laguna Niguel, CA (US); Jay A. Lenker, Laguna Beach, CA (US)

Correspondence Address: Robert D. Buyan;STOUT, UXA, BUYAN & MULLINS, LLP, Suite #300, 4 Venture, Irvine, CA, 92618, US

(73) Assignee: Microvention, Inc.

(21) Appl. No.: 10/726,135

(22) Filed: Dec. 1, 2003

Related U.S. Application Data
(62) Division of application No. 09/906,415, filed on Jul. 16, 2001, now abandoned.

(51) Int. Cl. A61F 2/06

(52) U.S. Cl. 623/1.21; 623/1.23; 623/1.34

(57) ABSTRACT

Methods and apparatus for treating or preventing endoleaks after an endovascular graft (e.g., a stent, tubular graft, stent-graft, coated stent, covered stent, intravascular flow modifier or other endovascular implant that affects, limits or prevents blood flow into a vascular defect such as an aneurysm, arterio-venous fistula, arterio-venous malformation, vessel wall perforation, etc.) has been implanted in the vasculature of a human or veterinary patient. An expansile polymeric material, such as a swellable polymer (e.g., a hydrogel), a flexible or elastomeric polymer foam (e.g. silicone, polyurethane, etc.) or a carrier member (e.g, a coil, filament, wire, etc) that carries a quantity of such expansile polymer is delivered into a perigraft space (i.e., space between the endovascular graft and the surrounding blood vessel wall) such that the polymeric material expands in situ to substantially fill the perigraft space or a portion thereof. The expansile polymeric material is delivered into he perigraft space through a catheter and/or cannula that is placed prior to, during or after the implantation of the endovascular graft. The invention includes an injector apparatus that is useable to deliver the expansile polymeric material through the wall of a previously implanted graft. After delivery into the perigraft space, the expanded polymeric material expands so as to fill all or an intended portion of the perigraft space in a manner that substantially prevents additional blood from leaking or flowing into such perigraft space. One type of blood-absorbing, porous, expansile polymeric material useable in this invention is a super-expansile hydrogel.

### FIELD OF THE INVENTION

The present invention relates generally to biomedical methods, materials and apparatus and more particularly to methods, materials and apparatus useable for treating or preventing leakage around endovascular grafts (i.e., “endoleaks”).

### BACKGROUND OF THE INVENTION

A. Treatment of Aneurysms by Endovascular Grafting:

Aneurysms are weakened areas in blood vessels which become distended forming a sac, and can rupture. Rupture of an aneurysm on a major artery can result in rapid hemorrhage and death if not promptly treated.

Aneurysms of the aorta are not uncommon and can be life threatening. Depending on which region(s) of the aorta is/are involved, the aneurysm may extend into areas of bifurcation (i.e., the inferior end of the aorta where it bifurcates into the iliac arteries) or segments of the aorta from which smaller “branch” arteries extend. In this regard, the various types of aortic aneurysms may be classified on the basis of the region(s) of aneurysmic involvement, as follows: and such aortic aneurysms can be classified in several categories as follows:
A. Thoracic Aortic Aneurysms: (aneurysms involving the portion of the aorta that extends through the chest cavity, including the ascending thoracic aorta and/or the aortic arch and sometimes also involving branch arteries which emanate therefrom (i.e., the subclavian arteries)
B. Thoracoabdominal Aortic Aneurysms: (aneurysms involving the portions of the aorta that extend into both the chest cavity and the abdominal cavity, including the descending thoracic aorta and branch arteries which emanate therefrom (i.e., thoracac intercostal arteries) and the abdominal aorta and branch arteries which emanate therefrom (i.e., renal, superior mesenteric, celiac and/or intercostal arteries).
C. Abdominal Aortic Aneurysms: (aneurysms involving the pararenal aorta and the branch arteries which emanate therefrom (i.e., the renal arteries) and/or aneurysms involving the infrarenal aorta with or without involvement of the iliac arteries.

The traditional “open surgical” approach to treating aortic aneurysms requires the formation of a large incision in the patents abdomen and/or chest, dissection and exposure of the aorta, surgical excision of the aneurysm and the anastomosis of a synthetic or natural tubular graft to the healthy aorta above and below the site of the excised aneurysm. Surgeries of this type are associated with significant risks of mortality or post-surgical complications such as infection, hemorrhage, renal failure, etc.

Endovascular grafting is a less invasive alternative to the traditional open surgical repair of aortic aneurysms. In endovascular grafting, a tubular graft is loaded onto or into a catheter, advanced into the aneurysmic vessel and caused to radially expand such that it becomes implanted within the aneurysmic segment of the aorta to form a prosthetic flow conduit through the aneurysm sac, and to effectively isolate weakened portion of the blood vessel wall from the hemodynamic forces and pressures of the flowing blood.

The prior art has included numerous endovascular grafts of varying design. Examples of endovascular grafting methods and devices include those described in the following U.S. Pat. No. 4,577,631 (Kreamer); U.S. Pat. No. 5,211,658 (Clouse); U.S. Pat. No. 5,219,355 (Parodi et al.); U.S. Pat. No. 5,316,023 (Palmaz et al.); U.S. Pat. No. 5,360,443 (Barone et al.); U.S. Pat. No. 5,425,765 (Tifenbrun et al.); U.S. Pat. No. 5,609,625; (Piplani et al.); U.S. Pat. No. 5,591,229 (Parodi et al.); U.S. Pat. No. 5,578,071 (Parodi); U.S. Pat. No. 5,571,173 (Parodi); U.S. Pat. No. 5,562,728 (Lazarus et al.); U.S. Pat. No. 5,562,726 (Chuter); U.S. Pat. No. 5,562,724 (Vorwerk et al.); U.S. Pat. No. 5,522,880 (Barone et al.); and U.S. Pat. No. 5,507,769 (Marin et al.), U.S. Pat. No. 5,984,955 (Wisselink).

The typical endovascular graft comprises a) a tube graft formed of flexible material such as expanded polytetrafluoroethylene (ePTFE) or woven polyester and b) a graft anchoring component (e.g., a stent, a frame, a series of wire rings, hooks, barbs, clips, staples, etc.) which operates to anchor the ends of the tube graft to healthy portions of the aorta at located above and below the aneurysm. The graft anchoring component may comprise a radially expandable stent or frame which is either incorporated into the body of the tubular graft or formed separately from the graft and deployed within the graft lumen. After the endovascular graft has been advanced into the aorta and maneuvered into its intended position, the graft anchoring component is radially expanded to exert outwardly-directed radial pressure against the surrounding aortic wall—thereby frictionally holding the graft in place. In some embodiments, hooks, barbs, or other projections formed on the graft anchoring device, will insert into the wall of the aorta to ensure that the graft will not move longitudinally after implantation. These radially expandable graft anchoring devices are generally classifiable as either a.) self-expanding or b) pressure-expandable. Graft anchoring devices of the “self-expanding” are usually formed of a resilient material (e.g., spring metal) or shape memory alloy which automatically expands from a radially collapsed configuration to a radially expanded configuration, when relieved of surrounding constraint (e.g., a surrounding tubular sheath or catheter wall). On the other hand, those of the “pressure-expandable” variety are typically formed of malleable wire or other plastically deformable material which will deform to a radially expanded configuration in response to the exertion of outwardly directed pressure by inflation of a balloon or actuation of another pressure-exerting apparatus positioned within the graft anchoring device.

B. Endoleaks Occurring After Implantation of Endovascular Grafts:

A major complication associated with the use of endovascular grafts to treat aortic aneurysms is the leakage of blood into the space between the tube graft and the aneurysmic aortic wall (hereinafter referred to as the “perigraft space”). These leaks are referred to as “endoleaks” and can result in the build up of arterial pressure within the perigraft space, with resultant catastrophic rupture of the aneurysm.

Endoleaks often result from a failure of the graft anchoring component to hold an end of the tube graft in firm coaptation with the adjacent aortic wall, allowing blood to leak into the perigraft space. Another cause of endoleaks is leakage of blood outwardly through the endovascular graft, such as through small holes that have been made in the wall of the graft for attachment of the graft anchoring device(s) or through iatrogenic perforations made in the wall of the graft during implantation.

Several ways have heretofore been proposed for redesigning or augmenting endovascular grafts to minimize the occurrence of endoleaks. For example, U.S. Pat. No. 6,015,431 (Thornton et al.) describes an endovascular graft that has a purportedly leak-resistant seal. Also, others have described methods for repairing endoleaks after they occur. For example, U.S. Pat. No. 6,203,779 B1 (Ricci et al.) describes methods for in situ sealing of endoleaks by injecting an adhesive polymer or prepolymer into the area where the endoleak is occurring in order to seal off the endoleak. While the methods described by Ricci et al. may be viable, such methods appear to have certain limitations or drawbacks. First, in order to place the injection catheter in a position where it can inject the adhesive polymer or prepolymer into the endoleak, it would first be necessary to precisely locate the endoleak. The performance of angiographic x-ray studies or other procedures to precisely locate the endoleak can be laborious and time consuming. Second, if the endoleak is diffuse and not specifically limited to definable location, it could be difficult or impossible to deliver the adhesive polymer or prepolymer to each location that would be required to effectively stop the endoleak. Third, it may be necessary for the adhesive polymer or prepolymer to adhere to the endovascular graft and to the adjacent blood vessel wall in order to effectively stop the endoleak and in the event such adhesion is not established or if such adhesion fails, the endoleak may re-occur. Fourth, Ricci et al. do not describe any way of using their adhesive polymer or prepolymer to prevent an endoleak before it occurs, but rather limit their description to ways of repairing endoleaks after they have occurred and after they have been located.

Also, U.S. Pat. No. 5,785,679 (Abolfathi et al.) describes methods and apparatus for treating aneurysms and arterio-venous fistulas (a-v fistulas) by first positioning a catheter having an inflatable balloon cuff within the affected blood vessel, inflating the cuff, percutaneously inserting a needle into the aneurysm sac (or a-v fistula) adjacent to the inflated balloon catheter cuff, injecting a synthetic molding material or biological hardening agent into the aneurysm sac (or a-v fistula), allowing such injected material or agent to harden, deflating the cuff of the balloon catheter and, finally, removing the balloon catheter such that a blood flow channel is formed through the hardened mass of synthetic molding material or biological hardening agent. This technique is purportedly not prone to endoleaks, because no endovasculartube graft remains in place and the injected material oragent is intended to completely fill the aneurysm or a-v fistula.

Also, U.S. Pat. No. 5,769,882 (Fogarty et al.) describes the disposition of an expansible sealing layer in a circumferential band about the exterior of an endovascular graft such that the sealing layer will form a seal between the graft and the adjacent vessel wall after the graft has been implanted. The sealing layer described by Fogarty et al. may be introduced prior to or simultaneously with the endovascular graft. Like the method of Ricci et al., the “sealing layer” described by Fogarty et al. can not be placed between the graft and the vessel wall after the endovascular graft has already been expanded and implanted. Rather, the Fogarty et al. approach is a preventative measure that is performed prior to or concurrently with the placement of the endovascular graft.

Also, PCT International Publication WO01/21108 A1 describes an expandable implant that substantially fills the aneurysmic space surrounding the endovascular graft. While PCT International Publication WO01/21108 A1 does describe methods for placing the implant within the aneurysmic space prior to or concurrently with the implantation of the aneurysm-bridging endovascular graft, it does not disclose any means or method(s) for placing the implant within the aneurysmic space after the endovascular graft has been implanted. Unfortunately, endoleaks are sometimes diagnosed days, weeks or even months after an aneurysm-bridging endovascular graft has been implanted and, in this regard, the system described in PCT International Publication WO01/21108 A1 may not be suitable for treating endoleaks in all cases, such as those wherein the endoleak is diagnosed after the endovascular graft has been placed.

Thus, in view of the above-discussed limitations and shortcomings, there remains a need in the art for the development of new materials, methods and devices capable of preventing or treating endoleaks a) without a need for knowledge of the precise location of the endoleak, b) without requiring adhesives to adhere to either the endovascular graft or the blood vessel wall and c) at any time, even after the endovascular graft has been implanted within the patient.

C. Biologically Compatible Hydrogels:

Generally, the term “hydrogel” refers generally to a polymeric material that is capable of absorbing water or other aqueous fluids and swelling without undergoing dissolution of the polymer matrix. Typically, as hydrogels swell, pores within their polymer matrices will increase in size. Because of these properties, hydrogels have heretofore been used as drug delivery materials for controlled release of drugs and as absorbent dressings or sponges for absorbing blood or other body fluids.

Typically, the rate at which a hydrogel swells when exposed to an aqueous fluid is limited by the rate at which the aqueous fluid can be absorbed into the hydrogel's glassy polymer matrix. Conventional dried hydrogels have relatively small pore sizes and thus exhibit relatively slow swelling. “Super-expansile” hydrogels capable of more rapid absorption of liquids and greater ratios of expansion than conventional hydrogels have been described in U.S. Pat. No. 5,750,585 (Park et al.) and PCT International Publication WO98/00000(Park). These super-expansile hydrogels generally comprise water swellable foam matrices formed as macroporous solids comprising a) a foam stabilizing agent and b) a polymer or copolymer of a free radical polymerizable hydrophilic olefin monomer crosslinked with c) about 0.1 to about 10% by weight of a multiolefin-functional crosslinking agent.

### SUMMARY OF THE INVENTION

The present invention provides methods for treating or preventing endoleaks after an endovascular graft has been implanted. As used hereafter and in the following claims, the term “endovascular graft” is to be broadly construed to literally include a stent, tubular graft, stent-graft, coated stent, covered stent, intravascular flow modifier or other endovascular implant that affects, limits or prevents blood flow into a vascular defect such as an aneurysm, arterio-venous fistula, arterio-venous malformation, vessel wall perforation, etc.) The method of the present invention generally comprises introducing an expansile polymeric material, such as a swellable polymer (e.g., a hydrogel) or a flexible or elastomeric polymer foam (e.g. silicone, polyurethane, etc.) into the perigraft space (the space between the endovascular graft and the surrounding blood vessel wall) such that the polymeric material expands in situ to substantially fill the perigraft space or a portion thereof. Thereafter, the expanded mass of polymeric material in the perigraft space substantially prevents additional blood from leaking or flowing into such perigraft space. One example of a blood-absorbing, porous, expansile polymeric material useable in this invention is a super-expansile hydrogel as described in U.S. Pat. No. 5,750,585 (Park et al.) and PCT International Publication WO98/00000(Park), the entireties of which are expressly incorporated herein by reference. Also, the expansile polymeric material may be in any suitable form (flowable liquid, solid, suspension, etc.) prior to and during its introduction into the perigraft space.

In accordance with the invention, the expansile polymeric material my be introduced into the perigraft space by any suitable means. In many applications, the expansible polymeric material will be introduced into the perigraft space through a cannula or tube. When the treatment is being administered after the endovascular graft has already been positioned and at least partially expanded, the cannula or tube may be advanced transluminally through the patient's vasculature to the location of the endovascular graft and, thereafter, into the perigraft space by a) advancement of the cannula through an opening in or by penetration through the wall of the graft or b) advancement of the cannula between the previously positioned endovascular graft and the adjacent blood vessel wall. Alternatively, a non-transluminal method may be employed wherein a needle or penetrator is used to penetrate percutaneously through the patients skin, through tissues underlying the skin and into the perigraft space and then the expansile polymeric material is introduced into the perigraft space through that needle or penetrator or through a separate cannula that has been advanced over or through that needle or penetrator.

Still further in accordance with the invention, solid particles (e.g., pellets, beads, dust, powder, pieces, etc.) of the expansile polymeric material may be introduced into the perigraft space through a cannula (e.g., needle, catheter, hypo-tube, etc.) and such solid particles may be suspended in a carrier fluid to facilitate their introduction through the cannula. After being introduced into the perigraft space, the expansile polymer particles will expand from their non-expanded state to their expanded state. In some applications, one or more solid particles of expansile polymeric material may be attached to a carrier member, such as a flexible or coiling filament or elongate member made of wire or other suitable material. For example, a plurality of solid pieces (e.g., pellets or small cylindrical pieces) of the expansile polymeric material may be mounted on or attached to an elongate coiling member at spaced-apart locations as described in U.S. Pat. No. 6,238,403(Greene, Jr. et al.), the entirety of which is expressly incorporated herein by reference. Or, a continuous covering or continuous mass of the expansile polymeric material may be disposed on all or a portion of the elongate coiling member as described in U.S. patent application Ser. No. 09/867,340, the entirety of which is expressly incorporated herein by reference. In embodiments where the expansile material is disposed on or associated with a carrier member, a disconnectable linkage may initially connect the carrier member to a delivery apparatus and, after the carrier member and accompanying expansile material have been introduced as desired into the perigraft space, the disconnectable connection may be severed or disconnected, thereby allowing the delivery apparatus to be withdrawn while leaving the carrier member and accompanying expansile material in place.

Still further in accordance with the invention, some embodiments of the expansile material will preferably expand to at least 5 times their original volumes (i.e., a ratio of pre-expansion volume to post-expansion volume of at least 1:5) and more preferably at least 10 times their original volumes (i.e., a ratio of pre-expansion volume to post-expansion volume of at least 1:5) when injected into the perigraft space.

Still further in accordance with the present invention, some embodiments of the expansile material, when in their fully expanded and/or cured states within the perigraft space, may be porous to allow blood or body fluid to permeate there into and/or to promote cellular in growth and/or post-implantation biological processes to occur, such as the gradual filling in of the perigraft space with natural granulation tissue. In these embodiments, the preferred size of pores formed in the expansile material, when it is fully expanded and cured, are about 50 to about 300 microns. Also, in these embodiments, the preferred porosity (i.e., the total volume of open pores relative to the total volume of the polymer is at least about 10% and preferably between about 20% and about 90%.

Still further in accordance with the invention, the expansile material may be biodegradable or non biodegradable.

Even further aspects of this invention will be come apparent to those of skill in the art upon reading of the detailed description of exemplary embodiments set forth herebelow.

### BRIEF DESCRIPTION OF THE DRAWINGS

FIGS. 1a-1e are diagrams that show, in step-by-step fashion, an example of one method of the present invention for treating an endoleak that has occurred following implantation of a bifurcated aorto-iliac endovascular graft in a human patient to treat an infra-renal aortic aneurysm that partially involves the patient's iliac arteries.

FIGS. 2a-2d are diagrams that show, in step-by-step fashion, an example of another method of the present invention for preventing the occurrence of an endoleak in a patient in whom an aortic endovascular graft has been implanted to treat an infra-renal aortic aneurysm.

FIG. 3 is a diagram of an example of yet another method of the present invention for treating an endoleak that has occurred following implantation of a aortic endovascular graft in a human patient to treat an aneurysm.

FIG. 4a is a side elevational view of the hand piece of a delivery catheter that is useable to introduce solid particles of expansile polymeric material or an embolization device that incorporates the expansible polymeric material, into a perigraft space in accordance with the present invention.

FIG. 4b is a side elevational view of the distal tip of the delivery catheter shown in FIG. 3 with its penetrating/injecting cannula withdrawn into the catheter lumen.

FIG. 4c is a side elevational view of the distal tip of the delivery catheter shown in FIG. 4 with its penetrating/injecting cannula advanced distally out of the catheter lumen.

FIG. 4d is a showing of a plurality of expansile polymeric material particles loaded into the delivery catheter of FIG. 3 for delivery into an aneurysm or perigraft space in accordance with the present invention.

FIG. 5 is a showing of an embolization device useable to fill an aneurysm in accordance with the present invention, such apparatus comprising a plurality of solid cylinders formed of expansile polymeric material mounted on a flexible carrier filament.

FIG. 5a is a sectional view through line 5a-5a of FIG. 5.

FIG. 5b is a sectional view through line 5b-5b of FIG. 5a.

FIG. 6A is a showing of another embolization device useable to fill an aneurysm in accordance with the present invention, such apparatus comprising a flexible carrier filament that is fully or partially covered with an expansile polymeric material.

FIG. 6B is a cross section through line 6B-6B of FIG. 6A.

FIG. 6C is partial longitudinal sectional view of the device of FIG. 6A.

FIG. 6D is a cross section through line 6B-6B of FIG. 6A after the expansile polymeric material has reached its expanded state.

FIG. 6E is partial longitudinal sectional view of the device of FIG. 6A, after the expansile polymeric material has reached its expanded state.

FIG. 7 is a diagram showing the manner in which a stabilized perigraft injector system of the present invention may be used to introduce an expansile polymeric material into the perigraft space following implantation of a bifurcated aorto-iliac endovascular graft in a human patient.

FIG. 7A is an enlarged view of segment 7A of FIG. 7.

### DETAILED DESCRIPTION OF THE INVENTION

The following detailed description and examples are provided for the limited purpose of illustrating exemplary embodiments of the invention and not for the purpose of exhaustively describing all possible embodiments of the invention.

Methods for Treating or Preventing Endoleaks

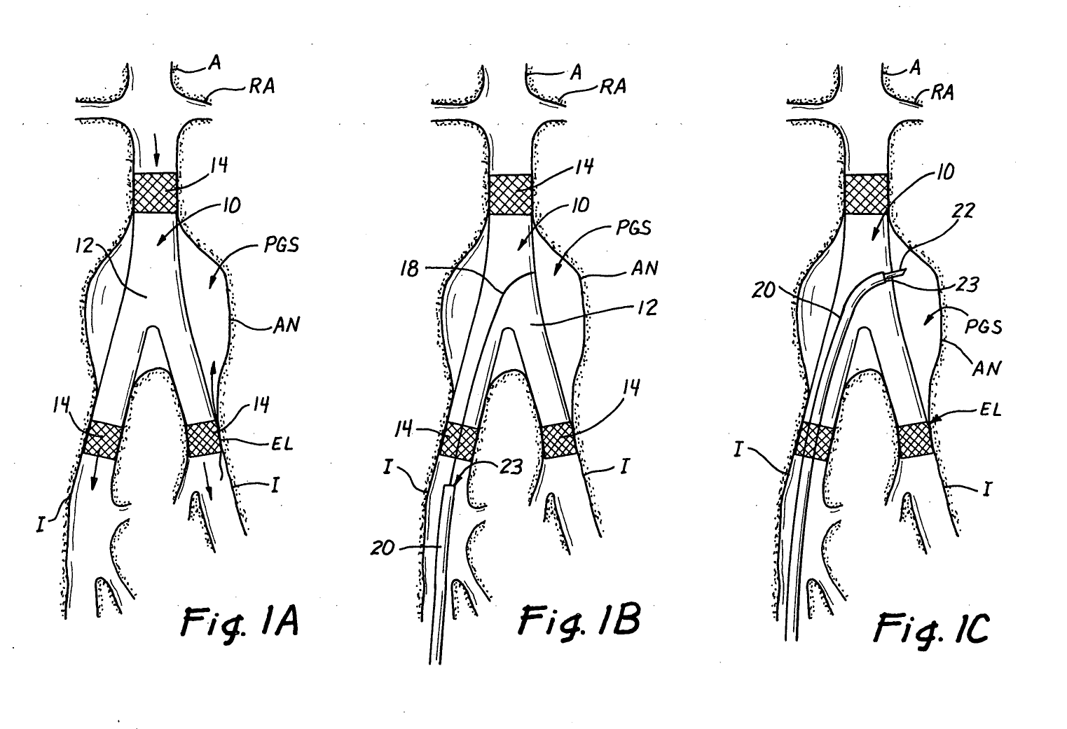

FIGS. 1A through 1E show one example of a method for treating an endoleak that has occurred in a bifurcated aorto-illiac endovascular graft 10 that has been implanted in a human patient to treat an abdominal aortic aneurysm AN that involved the infrarenal aorta A and portions of the illiac arteries I. In this example, the endoleak has resulted from less than adequate coaptation or sealing between the graft anchoring device 14 at the end of one of the bifurcated legs of the endovascular graft 10 and the wall of the patient's left iliac artery I. Initially, as shown in FIG. 1B, a guidewire 18 is inserted into the patient's right femoral artery and the guidewire 18 is advanced, using well known technique, through the right iliac leg of the graft 10 and into the main aortic portion of the graft 10. A catheter 20 is advanced over the guidewire to a position where the distal outlet opening 23 of the catheter 20 is directed at the wall of the graft 10 as shown in FIG. 1C. A hollow penetrator cannula 22 having a sharpened distal tip is then advanced out of the distal end opening 23 of the catheter 20 and through the wall of the graft into the perigraft space PGS, as also shown in FIG. 1C.

Thereafter, as shown in FIG. 1D, the expansile polymeric material 30 is introduced, while in its non-expanded state, through the lumen of the penetrator cannula 22 and into the perigraft space PGS. After being introduced into the perigraft space PGS, the expansile polymeric material 30 expands to its expanded state so as to substantially fill the aneurysmic sac in the manner shown in FIG. 1E.

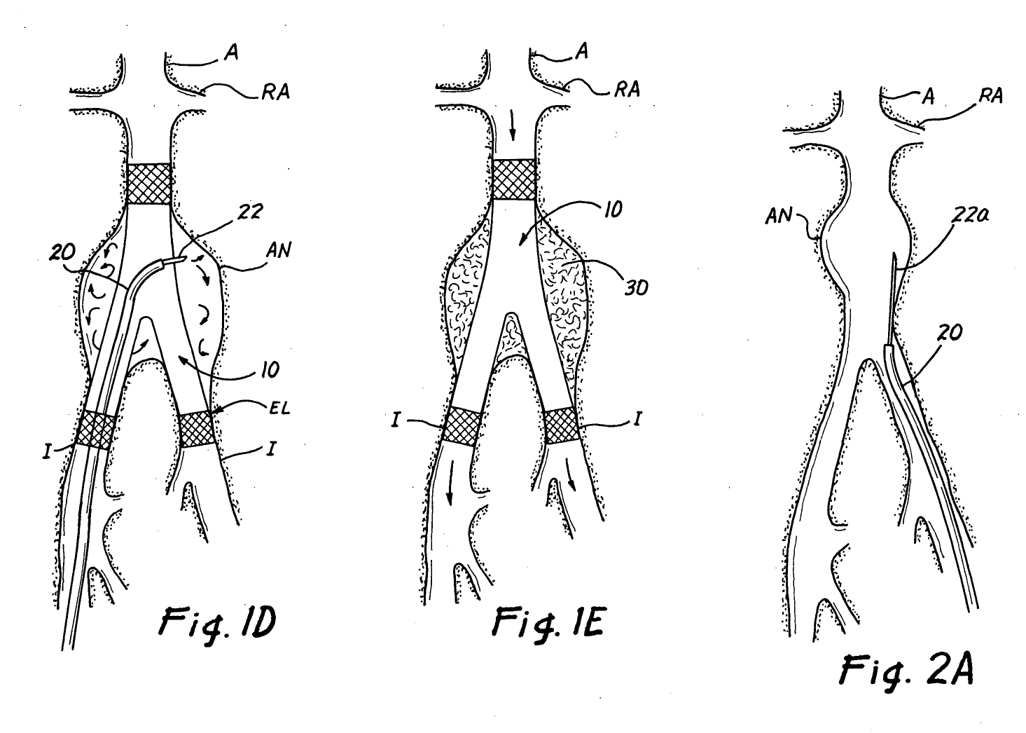

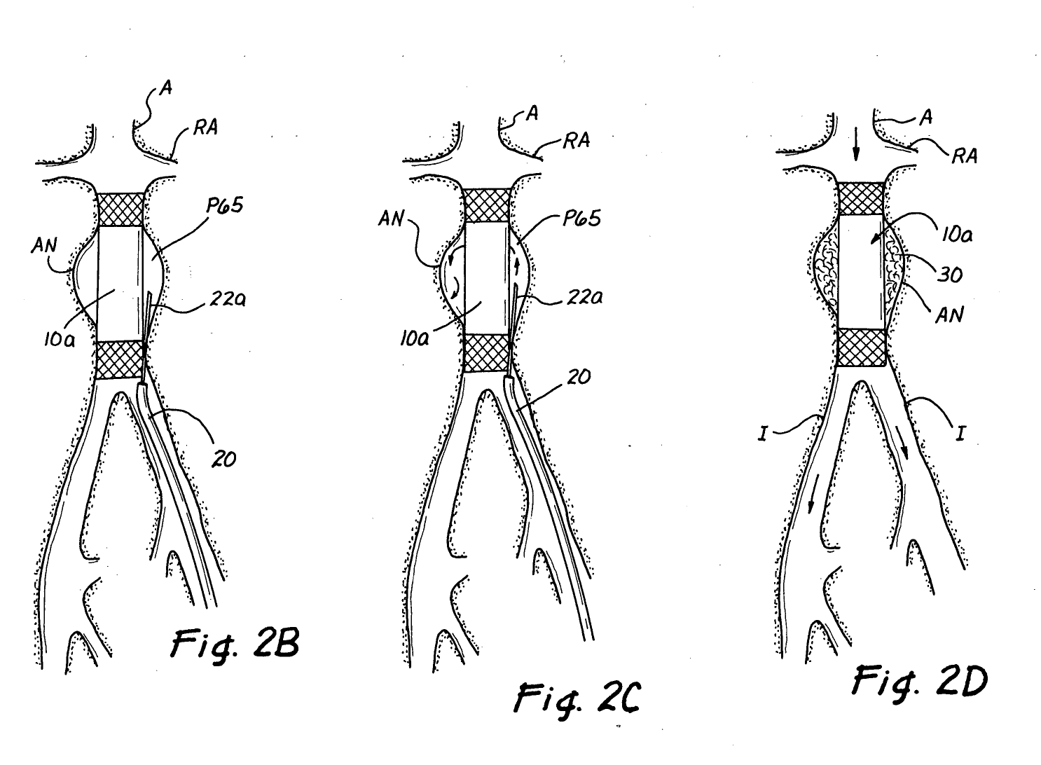

Another example of a method according to the present invention is shown in FIGS. 2A-2D. In this example, the aneurysm AN involves only the infrarenal abdominal aorta A and does not extend into the iliac arteries I. As shown in FIG. 2A, a catheter 20 is percutaneously inserted into a femoral artery and advanced to a position where the distal end of the catheter 20 is located within the aorta slightly inferior to the aneurysm. A blunt tipped cannula 22A is then advanced out of the end of the catheter 20, into the aneurysmic portion of the aorta. As shown in FIG. 2B, a straight endovascular graft 10a is then introduced, radially expanded and implanted, in accordance with technique well known in the art. When so implanted, the graft 10a bridges or extends through the aneurysm A and the graft anchoring devices 14a are in substantial coaptation with the healthy aortic wall above and below the aneurysm. The blunt tipped cannula 22a is captured between the inferior end of the graft 10a and the aorta wall, as shown. Preferably, the blunt tipped cannula 22a will be formed of metal hypotubing or plastic tubing that is sufficiently strong and crush resistant to avoid substantial collapsing or closing of its lumen when it is compressed between the adjacent graft anchoring device 14a and the aorta wall, as shown in FIG. 2B. Thereafter, as shown in FIG. 2C, the expansile polymeric material 30 is then injected through the catheter 20, through the lumen of the cannula 22A, and into the perigraft space PGS. After being introduced into the perigraft space PGS, the expansile polymeric material 30 expands to its expanded state so as to substantially fill the aneurysm sac. The catheter 20 and cannula 22 are then removed, leaving the graft 10a and expanded polymeric material 30 in place, in the manner shown in FIG. 2D.

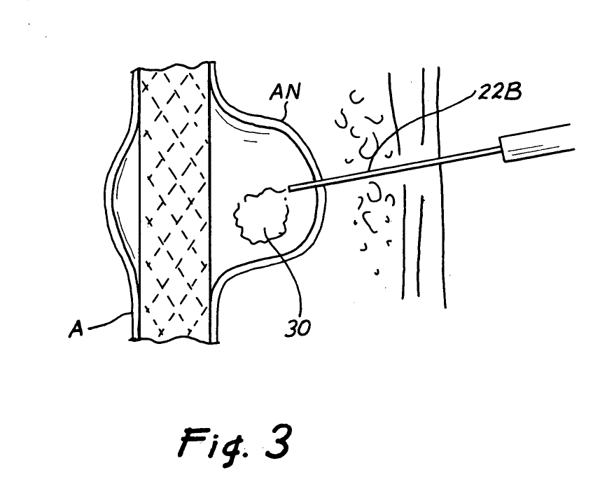

FIG. 3 shows an example of yet another method for carrying out the present invention, wherein the expansile polymeric material is injected into the perigraft space PGS through a cannula 20B that has been non-transluminally inserted through adjacent tissues and into the aneurysm sac. In this example, an abdominal aortic aneurysm A has been treated by placement of an endovascular graft 10 within the aorta. To treat an existing endoleak or to prevent aneurysm rupture or other complication that could arise from a subsequently occurring endoleak, it is desired to introduce an expansile polymeric material 30 into the perigraft space PGS within the aneurysm A. As shown in FIG. 3, the cannula 20B is inserted percutaneously into the patient's body, typically on the flan or side of the patient's back, and is advanced through the skin, muscle and other intervening tissues to a position where the distal end of the cannula 20B is positioned within the perigraft space PGS, within the aneurysm A. In applications where specific guidance of the cannula is desired to avoid damage to organs or critical anatomical structures, or for other reasons, the insertion and advancement of the cannula 20B may be carried out under radiographic guidance or with the use of steriotaxis as known in the art, examples of such radiographic guidance and/or stereotaxis instruments and methods being found in United States patent Nos. described in U.S. Pat. Nos. 4,733,661; 4,930,525 and 5,196,019, 5,053,042 and include those commercially available from various sources including the AccuPlace™ needle guide (In-Rad Corporation, Kentwood Mich.), the Bard CT Guide#550000 (C. R. Bard, Inc., Murray Hill, N.J.), the Picker Venue™ (Picker Corp., Cleveland, Ohio); and the Toshiba Aspire™ CT-fluoroscopy system (Toshiba America Medical Systems, Tustin, Calif.). Alternatively, the cannula 20B may be inserted and advanced with the aid of electro-anatomical mapping and/or guidance devices and methods, examples of which are found in U.S. Pat. Nos. 5,647,361; 5,820,568; 5,730,128; 5,722,401; 5,578,007; 5,558,073; 5,465,717; 5,568,809; 5,694,945; 5,713,946; 5,729,129; 5,752,513; 5,833,608; 5,935,061; 5,931,818; 6,171,303; 5,931,818; 5,343,865; 5,425,370; 5,669,388; 6,015,414; 6,148,823 and 6,176,829 and are commercially available as the Carto™ or NOGA™ system available from Biosense-Webster, Inc., a Johnson & Johnson Company, Diamond Bar, California and/or other systems available from Cardiac Pathways Corporation, 995 Benicia Avenue, Sunnyvale, Calif. and/or Stereotaxis, Inc., 4041 Forrest Park Avenue, St. Louis, Mo., or modifications thereof.

After the distal tip of the cannula 20B has been positioned within the perigraft space PGS, the expansile polymeric material 30 is injected through the cannula and into the perigraft space PGS, where it expands to substantially fill the aneurysm sac.

The Expansile Polymeric Material

The expansile polymeric material may comprise a hydrogel. Preferable hydrogels include a biocompatible, macroporous, hydrophilic hydrogel foam material as described in U.S. Pat. No. 5,570,585 (Park et al.), the entirety of which is expressly incorporated herein by reference as well as other hydrogels that undergo controlled volumetric expansion in response to changes in such environmental parameters as pH or temperature. An example of one such hydrogel that undergoes controlled volumetric expansion in response to changes in is environment is described in U.S. patent application Ser. No. 09/867,340, the entirety of which is expressly incorporated herein by reference. These pH responsive hydrogels are prepared by forming a liquid mixture that contains (a) at least one monomer and/or polymer, at least a portion of which is sensitive to changes in an environmental parameter; (b) a cross-linking agent; and (c) a polymerization initiator. If desired, a porosigen (e.g., NaCl, ice crystals, or sucrose) may be added to the mixture, and then removed from the resultant solid hydrogel to provide a hydrogel with sufficient porosity to permit cellular ingrowth. The controlled rate of expansion is provided through the incorporation of ethylenically unsaturated monomers with ionizable functional groups (e.g., amines, carboxylic acids). For example, if acrylic acid is incorporated into the crosslinked network, the hydrogel is incubated in a low pH solution to protonate the carboxylic acids. After the excess low pH solution is rinsed away and the hydrogel dried, the hydrogel can be introduced through a microcatheter filled with saline at physiological pH or with blood. The hydrogel cannot expand until the carboxylic acid grous deprotonate. Conversely, if an amine containing monomer is incorporated into the crosslinked network, the hydrogel is incubated in a high pH solution to deprotonate amines. After the excess high pH solution is rinsed away and the hydrogel dried, the hydrogel can be introduced through a microcatheter filled with saline at physiological pH or with blood. The hydrogel cannot expand until the amine groups protonate.

More specifically, in a preferred formulation of the hydrogel, the monomer solution is comprised of ethylenically unsaturated monomers, an ethylenically unsaturated crosslinking agent, a porosigen, and a solvent. At least a portion, preferably about 10% to about 50%, and more preferably about 10% to about 30%, of the monomers selected must be pH sensitive. The preferred pH sensitive monomer is acrylic acid. Methacrylic acid and derivatives of both acids will also impart pH sensitivity. Since the mechanical properties of hydrogels prepared exclusively with these acids are poor, a monomer to provide additional mechanical properties should be selected. A preferred monomer for providing mechanical properties is acrylamide, which may be used in combination with one or more of the above-mentioned pH sensitive monomers to impart additional compressive strength or other mechanical properties. Preferred concentrations of the monomers m the solvent range from 20% w/w to 30% w/w.

The crosslinking agent can be any multifunctional ethylenically unsaturated compound, preferably N,N′-methylenebisacrylamide. If biodegradation of the hydrogel material is desired, a biodegradable crosslinking agent should be selected. The concentrations of the crosslinking agent in the solvent should be less than about 1% w/w, and preferably less than about 0.1% w/w.

The porosity of the hydrogel material is provided by a supersaturated suspension of a porosigen in the monomer solution. A porosigen that is not soluble in the monomer solution, but is soluble in the washing solution can also be used. Sodium chloride is the preferred porosigen, but potassium chloride, ice, sucrose, and sodium bicarbonate can also be used. It is preferred to control the particle size of the porosigen to less than about 25 microns, more preferably less than about 10 microns. The small particle size aids in the suspension of the porosigen in the solvent. Preferred concentrations of the porosigen range from about 5% w/w to about 50% w/w, more preferably about 10% w/w to about 20% w/w, in the monomer solution. Alternatively, the porosigen can be omitted and a non-porous hydrogel can be fabricated.

The solvent, if necessary, is selected based on the solubilities of the monomers, crosslinking agent, and porosigen. If a liquid monomer (e.g. 2hydroxyethyl methacrylate) is used, a solvent is not necessary. A preferred solvent is water, but ethyl alcohol can also be used. Preferred concentrations of the solvent range from about 20% w/w to about 80% w/w, more preferably about 50% w/w to about 80% w/w.

The crosslink density substantially affects the mechanical properties of these hydrogel materials. The crosslink density (and hence the mechanical properties) can best be manipulated through changes in the monomer concentration, crosslinking agent concentration, and solvent concentration. The crosslinking of the monomer can be achieved through reduction-oxidation, radiation, and heat. Radiation crosslinking of the monomer solution can be achieved with ultraviolet light and visible light with suitable initiators or ionizing radiation (e.g. electron beam or gamma ray) without initiators. A preferred type of crosslinking initiator is one that acts via reduction-oxidation. Specific examples of such red/ox initiators that may be used in this embodiment of the invention are ammonium persulfate and N,N,N′,N′-tetramethylethylenediamine.

After the polymerization is complete, the hydrogen is washed with water, alcohol or other suitable washing solution(s) to remove the porosigen(s), any unreacted, residual monomer(s) and any unincorporated oligomers. Preferably this is accomplished by initially washing the hydrogel in distilled water.

As discussed above, the control of the expansion rate of the hydrogel is achieved by protonation/deprotonaton of the ionizable functional groups present on the hydrogel network. Once the hydrogel has been prepared and the excess monomer and porosigen have been washed away, the steps to control the rate of expansion can be performed.

In embodiments where pH sensitive monomers with carboxylic acid groups have been incorporated into the hydrogel network, the hydrogel is incubated in a low pH solution. The free protons in the solution protonate the carboxylic acid groups on the hydrogel network. The duration and temperature of the incubation and the pH of the solution influence the amount of control on the expansion rate. Generally, the duration and temperature of the incubation are directly proportional to the amount of expansion control, while the solution pH is inversely proportional. It has been determined that the water content of the treating solution also affects the expansion control. In this regard, the hydrogel is able to expand more in the treating solution and it is presumed that an increased number of carboxylic acid groups are available for protonation. An optimization of water content and pH is required for maximum control on the expansion rate. After the incubation is concluded, the excess treating solution is washed away and the hydrogel material is dried. The hydrogel treated with the low pH solution has been observed to dry down to a smaller dimension than the untreated hydrogel. This is a desired effect since delivery of these hydrogel materials through a microcatheter is desired.

In embodiments where pH sensitive monomers with amine groups were incorporated into the hydrogel network, the hydrogel is incubated in high pH solution. Deprotonation then occurs on the amine groups of the hydrogel network at high pH. The duration and temperature of the incubation, and the pH of the solution, influence the amount of control on the expansion rate. Generally, the duration, temperature, and solution pH of the incubation are directly proportional to the amount of expansion control. After the incubation is concluded, the excess treating solution is washed away and the hydrogel material is dried.

Examples of other biodegradable, expansile hydrogels that may be used in this invention include, but are not necessarily limited to those described in U.S. Pat. No. 5,162,430 (Rhee et al.), U.S. Pat. No. 5,410,016 (Hubbell et al.), U.S. Pat. No. 5,990,237 (Bentley et al.), U.S. Pat. No. 6,177,095 (Sawhney et al.), U.S. Pat. No. 6,184,266 B1 (Ronan et al.), U.S. Pat. No. 6,201,065 B1 (Pathak et al.), U.S. Pat. No. 6,224,892 B1 (Searle), U.S. Pat. No. 5,980,550 (Eder et al.) and PCT International Patent Publication Nos. WO 00/44306 (Murayama et al.), WO 00/74577 (Wallace et al.).

The expansile polymeric material, whether a hydrogel or other type of polymer, may be mixed with a carrier fluid to facilitate delivery into the body. In cases where the expansile polymeric material is in the form of solid pellets or particles, those pellets or particles may be suspended in a liquid carrier, such as saline, polyethylene glycol or a radiographic contrast medium. Alternatively, one or more solid pieces of the expansible polymeric material me be formed, mounted on or attached to a carrier member to facilitate introduction of the polymeric material into the aneurysm sac.

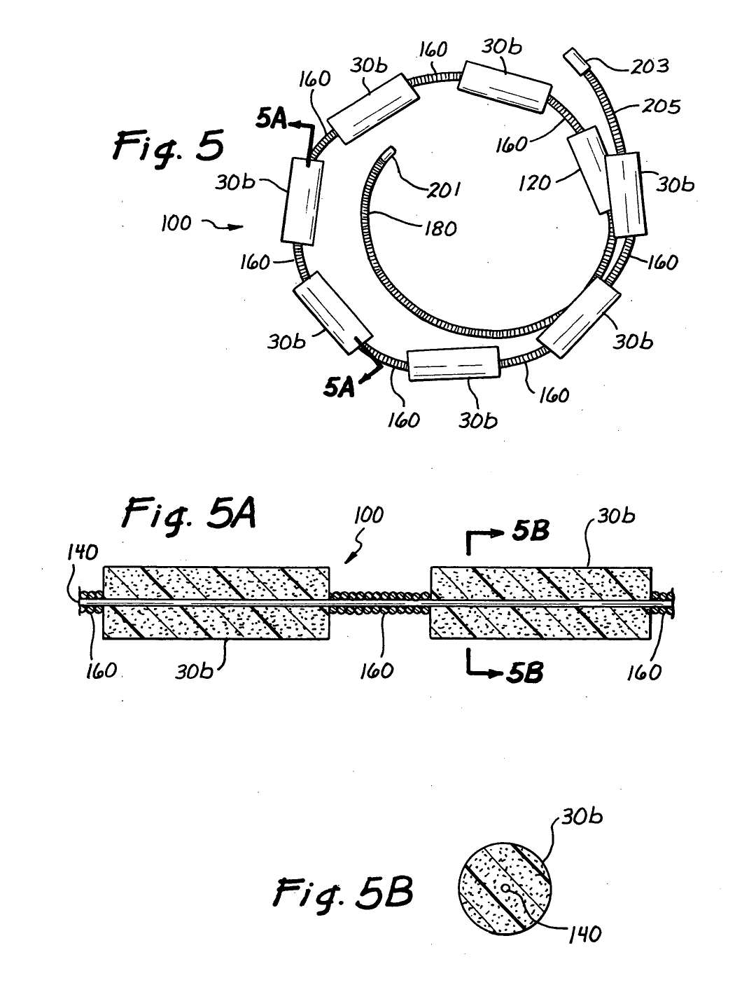

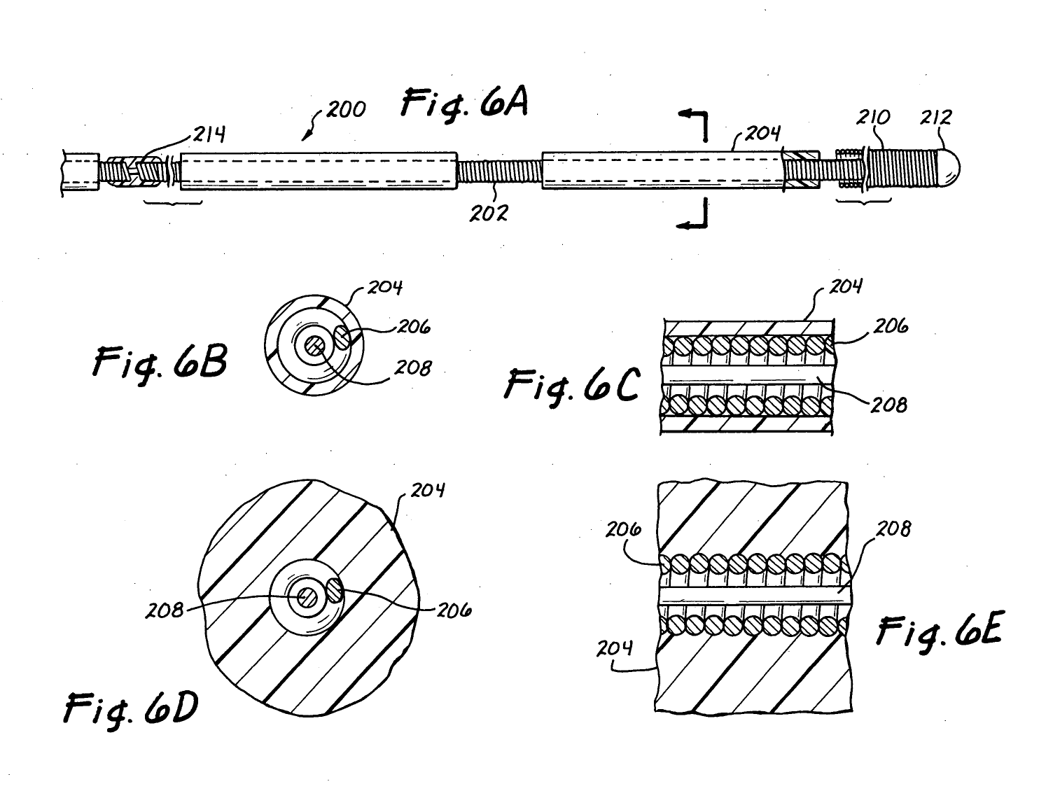

FIGS. 5 through 6E show examples of embodiments where a solid expansile polymeric material is disposed on a coiled carrier filament to form an implantable embolizing device 100 or 200 that comprises the expansile polymer.

In the particular example shown in FIGS. 5-5B, the embolization device 100 comprises a plurality of embolizing bodies, each configured as a substantially cylindrical pellet 120, located at spaced intervals along a filamentous carrier 140. The number of pellets 120 will vary, depending on the length of the carrier 140, which, turn, will depend on the size of the aneurysm sac to be embolized. The carrier member 140 comprises plurality of highly flexible coil spacers 160, each of which is disposed between and separates a pair of pellets 12. The carrier 140 has a distal portion on which is carried a relatively long distal coil segment 18 that is retained in place by a distal retention member 201. The carrier 140 has a proximal portion on which is carried a relatively long proximal microcoil segment 203. The proximal end of the device 100 is terminated by a hydrogel linkage element 203, to be described below. The spacers 160, the distal coil segment 180, and the proximal coil segment 205 are all highly flexible, and they are preferably made of platinum or platinum/tungsten wire, which has the advantages of being biocompatible and radiopaque. The pellets 120 are non-releasably carried on the carrier 140. They may be fixed in place on the filamentous carrier 140, either mechanically or by a suitable biocompatible, water-insoluble adhesive, or they may be simply strung loosely on the carrier 140 between successive spacers 160.

Another suitable material for the pellets 120 is a porous hydrated polyvinyl alcohol (PVA) foam gel prepared from a polyvinyl alcohol solution in a mixed solvent consisting of water and a water-miscible organic solvent, as described, for example, in U.S. Pat. No. 4,663,358 (Hyon et al.), the disclosure of which is incorporated herein by reference. Other suitable PVA structures are described in U.S. Pat. No. 5,823,198 (Jones et al.) and U.S. Pat. No. 5,258,042 (Mehta), the entireties of which are also expressly incorporated herein by reference. Another suitable material is a collagen foam, of the type described in U.S. Pat. No. 5,456,693 (Conston et al.), the entirety of which is also expressly incorporated herein by reference. Still another suitable material is PHEMA, as discussed in the references cited above. See, e.g., Horák et al., and Rao et al., supra.

The preferred foam material, as described in the above-referenced patent to Park et al., has a void ratio of at least about 90%, and its hydrophilic properties are such that it has a water content of at least about 90% when fully hydrated. In the preferred embodiment, each of the embolizing micropellets 12 has an initial diameter of not more than about 0.5 mm prior to expansion in situ, with an expanded diameter of at least about 3 mm. To achieve such a small size, the micropellets 120 may be compressed to the desired size from a significantly larger initial configuration. The compression is performed by squeezing or crimping the micropellets 120 in a suitable implement or fixture, and then “setting” them in the compressed configuration by heating and/or drying. Each of the micropellets 120 is swellable or expansible to many times (at least about 25 times, preferably about 70 times, and up to about 100 times) its initial (compressed) volume, primarily by the hydrophilic absorption of water molecules from an aqueous solution (e.g., resident blood plasma and/or injected saline solution), and secondarily by the filling of its pores with blood. Also, the micropellets 120 may be coated with a water-soluble coating (not shown), such as a starch, to provide a time-delayed expansion. Another alternative is to coat the micropellets 120 with a temperature-sensitive coating that disintegrates in response to normal human body temperature. See, e.g., U.S. Pat. No. 5,120,349 (Stewart et al.) and U.S. Pat. No. 5,129,180 (Stewart), the entireties of which are incorporated herein by reference.

The foam material of the embolizing pellet 120 may advantageously be modified, or provided with additives, to make the device 100 visible by conventional imaging techniques. For example, the foam can be impregnated with a water-insoluble radiopaque material such as barium sulfate, as described by Thanoo et al., “Radiopaque Hydrogel Microspheres”, J. Microencapsulation, Vol. 6, No. 2, pp. 233-244 (1989). Alternatively, the hydrogel monomers can be copolymerized with radiopaque materials, as described in Horak et al., “New Radiopaque PolyHEMA-Based Hydrogel Particles”, J. Biomedical Materials Research, Vol. 34, pp. 183-188 (1997).

It will be appreciated that in any embodiment of the invention, the expansile polymeric material may further include, contain, comprise or incorporate a medicament (e.g., drug, biological, gene, gene therapy preparation, diagnostic agent, imageable contrast material, growth factor, other biological factor, peptide or other bioactive compound, therapeutic or diagnostic substance) to cause a desired medicament effect (a therapeutic, diagnostic, pharmacological or other physiological effect) in the patient.

The filamentous carrier 140 is preferably a length of nickel/titanium wire, such as that marketed under the trade name “Nitinol”. Wire of this alloy is highly flexible, and it has an excellent “elastic memory”, whereby it can be formed into a desired shape to which it will return when it is deformed. In a preferred embodiment of the invention, the wire that forms the carrier 140 has a diameter of approximately 0.04 mm, and it is heat-treated to form a multi-looped structure that may assume a variety of three-dimensional shapes, such as a helix, a sphere, or an ovoid (as disclosed, for example, in U.S. Pat. No. 5,766,219 (Horton), the disclosure of which is incorporated herein by reference). Preferably, the intermediate portion of the carrier 14 (i.e., the portion that includes the micropellets 12) and the proximal portion (that carries the proximal microcoil segment 22) are formed into loops having a diameter of approximately 6 mm, while the distal portion (that carries the distal microcoil segment 18) may have a somewhat greater diameter (e.g., approximately 8-10 mm). The carrier 14 may be formed of a single wire, or it may be formed of a cable or braided structure of several ultra-thin wires.

In another embodiment, the carrier 140 may be made of a thin filament of a suitable polymer, such as a PVA, that is formed in a looped structure. The polymer may be impregnated with a radiopaque material (e.g., barium sulfate or particles of gold, tantalum, or platinum), or it may enclose a core of nickel/titanium wire. Alternatively, the carrier 14 may be constructed as a “cable” of thin polymer fibers that includes fibers of an expansile polymer, such as polyvinyl alcohol (PVA), at spaced intervals to form the micropellets 120.

Still another alternative construction for the carrier 140 is a continuous length of microcoil. In such an embodiment, the micropellets 120 would be attached at spaced intervals along the length of the carrier 140.

The hydrogel linkage element 203 may be made of the same material as the pellets 120. Indeed, the most proximal of the micropellets 120 may function as the linkage element 203.

Another embodiment of an embolizing device 200 that incorporates the expansile polymeric material is shown in FIGS. 6A-6E. In this embodiment, the embolization device 200 comprises an elongate, flexible, filamentous carrier 202 which is substantially covered by an embolizing element 204 formed of a suitable expansile polymeric material such as any of those described hereabove. The embolizing element 204 is non-releasably carried on the elongate carrier member 202. The carrier member 202 is preferably formed from a continuous, hollow coil 106, made from a suitable metal such as platinum, gold, tungsten, or tantalum, or a metallic alloy, such as stainless steel or Nitinol. Of these materials, platinum and Nitinol are preferred. The coil is formed of tightly packed convolutions, so that there is little or no spacing between adjacent convolutions of the coil. The carrier 202 may also include a filamentous core 208 extending axially through the coil 206. The core 208 is a thin metal wire, preferably made of a shape memory metal such as Nitinol. The device 200 includes a distal portion comprising an outer coil 210 coaxially surrounding the coil 206, and terminating in a rounded distal tip 212. A hydrogel linkage element (not shown), of the type described in relation to the embodiment shown in FIGS. 5-5D and described above may advantageously be provided at the proximal end of the carrier member 202.

The carrier 202 may, alternatively, be made of any of the materials described above with respect to the carrier of the first preferred embodiment. While it is preferably in the configuration of a coil, it may also be formed as a single strand of metal wire or polymeric filament, or as a multi-strand braid or cable of metal wire or polymeric filament. The carrier should have a column strength sufficient to allow it to be pushed through a microcatheter, as mentioned above.

Further description and some possible variations/modifications of this embodiment of the embolization device 200 are shown and described in co-pending U.S. patent application Ser. No. 09/867,340, the entirety of which is expressly incorporated herein by reference.

A Device for Delivering the Expansile Polymeric Material Into the Perigraft Space Within the Aneurysm Sac:

The expansile polymeric material, when in the form of a flowable liquid or suspension of particles or pellets, may be introduced into the perigraft space through any suitable cannula 22, 22A, 22B, including needles, hypotube, catheter or other tubular conduits. When, however, the expansile polymeric material is incorporated into an implantable embolization device such as the devices 100, 200 described above, it is desirable to use a more specialized delivery cannula for delivering the embolization device into the perigraft space.

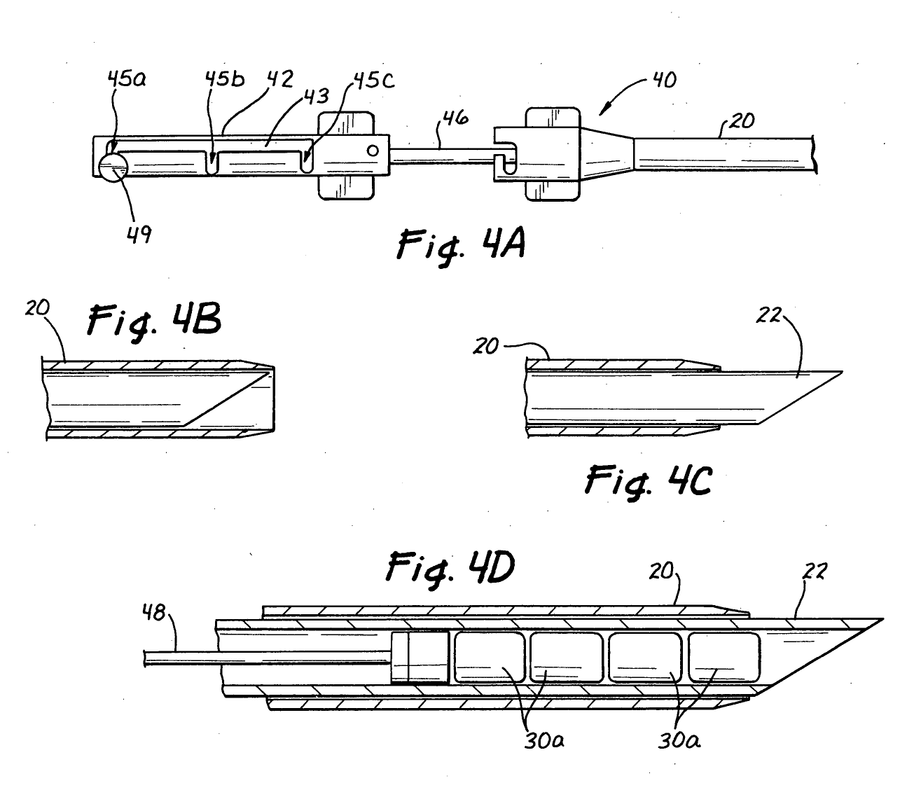

One example of a delivery device 40 useable for delivering an elongate embolization coil or device (such as the embolization devices 100, 200 described above) is shown in FIGS. 4A-4d. This delivery device 40 comprises a catheter 20 that has a delivery cannula 22 coaxially disposed within and slidably advanceable from the lumen of the catheter 20.

A pusher rod 48 is inserted into the proximal portion of the delivery cannula 22. A handpiece is formed on the proximal end of the cannula. When the handpiece is advanced in the distal direction, the distal end of the delivery cannula 22 advances out of the distal end of the catheter 20 as shown in FIG. 4C. When the handpeice 42 is retracted in the proximal direction, the distal tip of the delivery cannula 22 is retracted into the lumen of the catheter 20 as shown in FIG. 4B.

A knob 49 is formed on the proximal end pusher member 48 and is advanceable and retractable within a track 43 formed on the handpiece 42. Advancement of the knob 49 in the distal direction will advance the pusher member 48 in the distal direction and retraction of the knob 49 in the proximal direction will cause the pusher member to retract in the proximal direction. Notches 45a, 45b and 45c are formed in the track to facilitate stopping and locking of the knob 49 in various partially advanced and fully advanced positions.

A series of pieces or pellets 30a of the expansile polymeric material may be positioned in the lumen of the delivery cannula 22, distal to the pusher member, as shown in FIG. 4d. As the pusher member 48 is advanced, the pellets 30a will be expelled from the distal end of the delivery cannula 22, into the perigraft space. Similarly, an embolization device 100, 200 that incorporates the expansile polymeric material may be placed in a substantially linear configuration and inserted into the lumen of the delivery cannula 22 distal to the pusher member 48 and advancement of the pusher member in the distal direction will expel the embolization device out of the distal end of the delivery cannula 22 and into the perigraft space. If biased to a coiled configuration, the embolization device 100, 200 may then assume its coiled configuration after it has been introduced into the perigraft space.

In some embodiments, the pellets 30a or embolization device 100, 200 may be attached to the pusher member 48 by a disconnectable (e.g., severable, separable, releasable or breakable) linkage so as not to become separated from the pusher member 48 until the linkage is severed. The severable linkage may comprise a tube having a plug member inserted in the distal end of the tube and attached to the embolization device such that, after the embolization device has been implanted in the perigraft space as desired, a fluid may be injected through the tube to propel the plug member out of the tube, thereby separating the embolization device from the tube. Examples of this type of disconnectable linkage are found in copending U.S. patent application Ser. No. 09/692,248 (Ferrera et al), the entirety of which is incorporated herein by reference. Alternatively, any other suitable type of disconnectable linkages may be used, including linkages that disconnect by either mechanical means, biodegradation, dissolution, electrolysis or by way of an electro-mechanical disconnection apparatus.

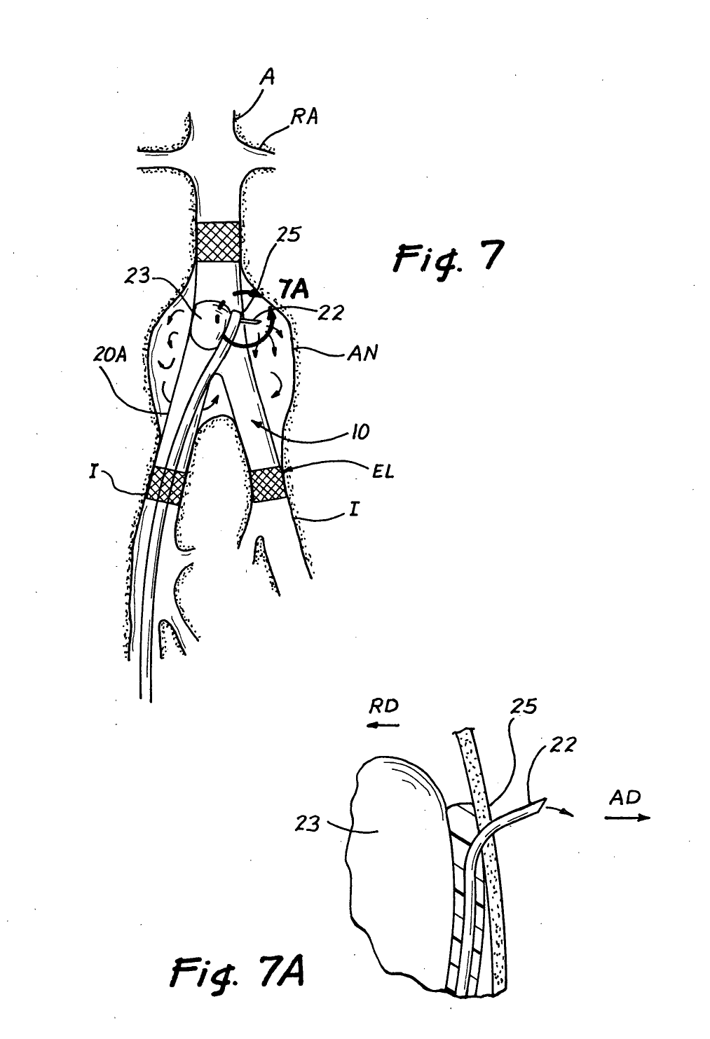

As shown in FIGS. 7 and 7A, in some embodiments, a stabilized catheter 20c may be used. This stabilized catheter has a stabilization member 63, such as an inflatable balloon or deployable lateral member, located adjacent the outlet port 25 through which the cannula 22 is advanced. This stabilization member 23 is deployed (e.g., the balloon is inflated) prior to and during the advancement of the cannula 22 through the wall of the endovascular graft 10, thereby preventing the catheter 20A from recoiling in a recoil direction RD that is generally opposite to the advancement direction AD in which the cannula 22 is advanced through the wall of the graft 10. This facilitates the desired penetration of the cannula through the wall of the graft 10 and into the perigraft space.

It will be appreciated that in any embodiment of the invention, the hydrogel may further include or incorporate a medicament (e.g., drug, biological, gene, gene therapy preparation, diagnostic agent, imageable contrast material, growth factor, other biological factor, peptide or other bioactive compound, therapeutic or diagnostic substance) to cause a desired medicament effect (a therapeutic, diagnostic, pharmacological or other physiological effect) in the patient. Examples of some of the types of medicaments that may be incorporated into the hydrogels of this invention are described in U.S. Pat. No. 5,891,192 (Murayama, et al.), U.S. Pat. No. 5,958,428 (Bhatnagar) and U.S. Pat. No. 6,187,024 (Boock et al.) and in PCT International Publication WO 01/03607 (Slaikeu et al.), the entireties of each such document being expressly incorporated herein by reference. Specifically, byway of example, the pellets 120 may optionally include bioactive or therapeutic agents to promote thrombosis, cellular ingrowth, and/or deposition of granulation tissue, healing, etc. See, e.g, Vacanti et al., “Tissue Engineering: The Design and Fabrication of Living Replacement Devices for Surgical Reconstruction and Transplantation,” The Lancet (Vol. 354, Supplement 1), pp. 32-34 (July, 1999); Langer, “Tissue Engineering: A New Field and Its Challenges,” Pharmaceutical Research, Vol. 14, No. 7, pp. 840 -841 (July, 1997); Persidis, “Tissue Engineering,” Nature Biotechnology, Vol. 17, pp. 508-510 (May, 1999).

The invention has been described herein with reference to certain examples and embodiments only. No effort has been made to exhaustively describe all possible examples and embodiments of the invention. Indeed, those of skill in the art will appreciate that various additions, deletions, modifications and other changes may be made to the above-described examples and embodiments, without departing from the intended spirit and scope of the invention as recited in the following claims. It is intended that all such additions, deletions, modifications and other changes be included within the scope of the following claims.

1-78. (Canceled)

79. A method for preventing leakage into a perigraft space between an endovascular graft that has been implanted in the lumen of a blood vessel of a human or veterinary patient and an adjacent portion of the blood vessel wall, said method comprising the steps of:
(A) providing a device comprising a solid member having expansile polymeric material disposed thereon, said expansile polymeric material being i) initially in a non-expanded state wherein a quantity of the polymeric material occupies a first volume and b) subsequently expandable to an expanded state wherein said quantity of the polymeric material occupies a second volume larger than the first volume and absorbs blood;
(B) inserting a cannula into a perigraft space between the endovascular graft and the blood vessel wall;
(C) introducing the device through the cannula and into the perigraft space while the expansile polymeric material is substantially in its non-expanded state;
(D) allowing the polymeric material to expand to its expanded stated within the perigraft space such that the device substantially fills the perigraft space.

80. A method according to claim 79 wherein i) the adjacent portion of the blood vessel wall is aneurysmic; ii) the endovascular graft is implanted within the blood vessel such that it extends through the aneurysmic portion of the blood vessel and defines a perigraft space between the graft and the aneurysmic wall of the blood vessel; and, iii) the device is introduced into the perigraft space where the expansile polymeric material expands to substantially fill the perigraft space.

81. A method according to claim 80 wherein the total volume of non-expanded expansile polymeric material introduced in Step C is predetermined to substantially fill the perigraft space after it has been allowed to expand in Step D.

82. A method according to claim 79 wherein the expansile polymeric material is radiopaque.

83. A method according to claim 82 wherein the expansile polymeric material is rendered radiopaque by the incorporation of radiopaque monomers.

84. A method according to claim 79 wherein the polymeric material expands to its expanded state when the pH of its environment is a physiological pH of about 7.4.

85. A method according to claim 79 wherein the polymeric material is in the form of pellets when introduced through the cannula.

86. A method according to claim 79 wherein the solid member is an elongate member.

87. A method according to claim 86 wherein the solid member is filamentous.

88. A method according to claim 86 wherein a plurality of pieces of the polymeric material are disposed at spaced-apart locations on said elongate solid member.

89. A method according to claim 88 wherein the device further comprises coil spacers disposed on said solid member between pieces of the expansile polymeric material.

90. A method according to claim 79 wherein the solid member is formed of platmium.

91. A method according to claim 79 wherein the solid member is formed of platinum and tungsten.

92. A method according to claim 79 wherein the solid member is formed of wire.

93. A method according to claim 79 wherein the solid member is formed of polymeric material.

94. A method according to claim 93 wherein the solid member is formed of a polymer filament.

95. A method according to claim 94 wherein the solid member is formed of a polyvinyl alcohol filament.

96. A method according to claim 79 wherein the solid member is biased to a coiled configuration.

97. A method according to claim 79 wherein the cannula is advanced through the lumen of a catheter.

98. A method according to claim 97 wherein the catheter is a microcatheter.

99. A method according to claim 98 wherein the microcatheter has a lumen of 0.005-0.050 inch diameter.

100. A method according to claim 79 wherein the device is initially attached to a delivery member by way of a detachable connection, said delivery member being useable to advance the device into the perigraft space, said detachable connection being thereafter detachable such that the delivery member may be retracted into the cannula while the device remains in the perigraft space.

101. A method according to claim 79 wherein the polymeric material expands more rapidly as the pH of its environment increases.

102. A method according to claim 79 wherein the polymeric material is a hydrogel.

103. A method according to claim 79 wherein the polymeric material is porous when in its expanded state.

104. A method according to claim 103 wherein the porous polymeric material, when substantially fully expanded, has pores of about 50-1000 microns in diameter.

105. A method according to claim 103 wherein the porosity of the polymeric material, when substantially fully expanded, is at least about 50%.

106. A method according to claim 103 wherein the porosity of the polymeric material, when substantially fully expanded, is between about 50% and about 95%.

107. A method according to claim 79 wherein the graft is implanted prior to performance of Step B.

108. A method according to claim 107 wherein Step B further comprises:
causing the distal end of the cannula to enter the perigraft space by penetrating through a portion of the graft.

109. A method according to claim 107 wherein Step B further comprises:
causing the distal end of the cannula to enter the perigraft space by advancing through tissue of the patient's body, through the wall of the blood vessel adjacent to the graft and into the perigraft space.

110. A method according to claim 107 wherein Step B further comprises:
passing a substantially hollow needle through tissues of the patient's body and through the wall of the blood vessel adjacent to the perigraft space; and,
advancing the cannula through the needle such that the distal end of the cannula enters the perigraft space.

111. A method according to claim 79 wherein the cannula is substantially rigid.

112. A method according to claim 79 wherein the cannula is substantially flexible.

113. A method according to claim 79 wherein the cannula comprises a metal tube.

114. A method according to claim 79 wherein the cannula comprises a plastic tube.

115. A method according to claim 79 wherein the method is performed after an endoleak has been detected as a means of treating the endoleak.

116. A method according to claim 79 wherein the method is performed before an endoleak has been detected as a means for preventing an endoleak from occurring.

117. A method according to claim 79 wherein Step B comprises:
advancing a catheter to a first position within the patient's vasculature; and,
advancing the cannula through the catheter to a second position wherein the distal end of the cannula is within the perigraft space.
